SEQUENCE LISTING (7 sequences)

SEQ ID NO: 1 - DNA, 3397 bp, Artificial sequence
  Description of the artificial sequence chemically pretreated human genomic DNA
ttatgtgttttaataaaaagtttaggataattggtaagagttttaatttgttagtggcgtgagttattattggttaaggttttttttttttttttttgagatagagttttgttttgttatttaggttggagtgtagtggtacgattttgggttattgtaattttcgttttttgggtttaagcgatttttttgttttagtttttcgagtagttgggattataggtgtgtgttattatgtttaggtaattttttgtatttttagtagagacggggttttattatgttagttaggatggtttcgattttttgattatgtgatttgtttgtttcggttttttaaagtgttgggattataggcgtgagttattgtatttagtcgtggtttaggttttattagtaaataaattatgaaaaattaggagaagtggataaagaataggaagaaagaattataagttaaaagaattgtaaaggaaaaattaggtagttttgagttttagttgtatagaaaaggtaggtgagatttgtaaagagaagtattttatttgtgttgggtttttagttgttaaagttttgggtaatattttcggtaggatgtttagggggaatttttgggtagaggtgaagggttttgttacgtgaattatttagttttattagttggtgatgaaatggtttataaagtatagattttgagtgttttgttagttttgggaattatttggagattgatatttttttacgtattatatagtggtagagaaggtaattattttttggttgttggtatttttagtggaagagtaaatattaagtgagagttattattttgaggtaggtagtattttaatatttatatatataataattttttaataattaattagtaatgatgttattatttttattttatagatgaaggaattgaggttttttgtttttagtaaaattagtagagttacggtttgaattttggtagtttgtttttagaggttgttttattttaggaatattattagttagtttaataagtagtttgtggaggatatagtatgttttttgttttggaattcggtatttttgagttataaatagtttattttttttattcggaaaaggaatggagaagttgggttagattatttttttttttttttttttttgagatagagttttgtttttgttgtttaggttggaatgtaatggtttgatttttgtttattgtaatttttattttttaggtttaagttatttttttgttttagttttttaagtagttgggattataggtatgacgtaattatgtatggttaattttggatttttagtagagattaaattttagtagagattagattttaggttttattatgttggttaggttggtttggaatttttgattttaggtgattagtttttttaagttttttaaagtgttgggattataggtgtgagcgtattacgttcggttaaagtatttttatataatgtggtatattgttaaataggaaagtttattggtggttcgatagaaatttttgtgtttttaagtatattttaaacgtgtttggaaagttattgagttaaagtgattgattttgaaggtataaattaatttagttattatttagttaataaatttattgtagattttattgttattttgtttttttgaaatttattttgttttaaatcgagattgttacgtaaaagtgaaatttagataaatttagaattttattattttgtggtttattttaaagatcgaaaaatgtttgggttaaattttaaataaaagtattaagttttgtaattaaaaattgaattaaaaatattaacgagagtgttttgtaaaattgaaatttaaataaatttataattatattaggtttggtttttgttgtttgttttaaagattgaaaaatatttgggttaaatttttaataaaaagattaagttttgtaattaaaatttaaatttaaaatattttattagtaaaattatttttaatttttgttgtatgtttgaaagtttttataataagatggataggaaaaaagtattttattaaaatttaaagagtgatgacgaattttttttttgtgtcggtttattaaattgtttaatggtaattacggataagtaaatgtttttatttcgaatcggttttgttttttaaacgaatgacgggtaatagagatcgaaaaagtgtatttttaggaattcgagagggacggtgaattgtagtgggatcgttaggaggggaggtttttgttttcgagtagaatttgggaggagagtcgttcggggagggcggtgggggttttagggtcgcggagggggtttttttagggttgggtcgtttttttttcgttcgtcgtaggtaggagcgcgggggatcgaaattttgtagtttcgtagcgttaggtagcgggtttgcgtttagcgagcggttttttagtttttgggaagatgcggattcgggacgttttcgtgagtttattgcgtttggttgatacgaggcgtttatagaataaagtaagggtttcggggagggcgcggtcgcggggttagcgcgtagatcgtttcggattcggatatcgtttgcgaggagcgtcgattagtcgggaagggttcgcgttaggcggcgttcgggtttcgtcggttagggtgagcgttcggttcgcgttcgcgttacgtcgcgcgttttttttttttttgcggcgggtcgagagataattttgttcgaggggtttcggcgttcgttttttacgcggtcgtattggaattcgtagtttttttcgggttttcggggcgattattttgtagtttcgttcgtttatatttgttttaggatcggcggttatttcggtcgtagcgcgaggcgcgattttttttttggggggtaagatggtaggtttcgggaaataaaggaaatttgggttcggtttttagcgattcgatgcgtgtgtggttcgcgtgtgtgtttcgggttttgtgtcggacgttgggtttgtgttagtttgttcgtagttcgtagtcgggcgggatatagtcggtattagtagtcggcggcgttcgtttatttgtagcggcgcgtcgttcggggggtttatcggttcgcgacgagagcggcggtggcggtggcggcggcggcggggggcgttggggaatcgacgcgggcgggaggcggcgggcggttggcgaggggagtcggggtgggcgggcggcgttacgttacggttattgtggtttttttggtatataaggtttcgtcggttcgtcgcgttttttattttgtttgcgttcgttcggagttagcggttttttaagtatttagtattttgttagacgcgtcgcgtatcgaacggaggggatatgggtagagtaatggtgg

SEQ ID NO: 2 - DNA, 3397 bp, artificial sequence
  Description of the artificial sequence chemically pretreated human genomic DNA
ttattattgttttgtttatgtttttttcgttcggtgcgcggcgcgtttagtaggatgttgggtgtttggagaatcgttggtttcgggcgggcgtaggtaaggtggggagcgcggcgagtcggcgagattttatatattaggaaagttataatagtcgtgacgtggcgtcgttcgtttatttcggtttttttcgttagtcgttcgtcgtttttcgttcgcgtcggttttttagcgtttttcgtcgtcgtcgttatcgttatcgtcgttttcgtcgcgggtcggtgagtttttcgggcggcgcgtcgttgtaggtgggcgggcgtcgtcggttgttggtatcggttgtatttcgttcggttgcgggttgcgggtaggttggtataggtttagcgttcggtataggattcggggtatatacgcgagttatatacgtatcggatcgttgggggtcgggtttaagttttttttgtttttcgggatttgttattttattttttaaaagaaaagtcgcgtttcgcgttgcggtcggggtagtcgtcggttttggagtaagtgtgggcgagcgagattgtaaaatgatcgtttcggggattcgagaggggttgcgaattttagtgcgatcgcgtggggggcgggcgtcgggatttttcgggtagggttatttttcggttcgtcgtagaggaaaggggaacgcgcggcgtggcgcggacgcgggtcggacgtttattttggtcgacgggattcgggcgtcgtttagcgcgaatttttttcggttggtcggcgtttttcgtaggcggtgttcgggttcggagcgatttgcgcgttggtttcgcggtcgcgtttttttcgaagtttttgttttgttttgtgagcgtttcgtgttagttaggcgtagtgagtttacgggggcgtttcgggttcgtatttttttaggagttggggagtcgttcgttgggcgtagattcgttgtttgacgttgcgaaattatagggtttcggtttttcgcgtttttgtttgcggcgggcgaggaggagacggtttaattttgaggggatttttttcgcggttttgaagtttttatcgtttttttcgagcggttttttttttaggttttattcgggggtaggaatttttttttttggcggttttattataatttatcgtttttttcgaatttttggggatatattttttcggtttttgttgttcgttattcgtttaaagagtagaatcggttcgagataaaggtatttatttattcgtagttgttattaggtaatttaatgaatcggtataaagaggagattcgttattattttttagattttggtggaatattttttttttatttattttattgtgaaaatttttaagtatataataaaagttgaaagtaattttattggtggagtattttgagtttaaattttaattgtaagatttaatttttttattgaaggtttagtttaagtgttttttagtttttaaagtaaatagtagagattaaatttagtatggttgtaaatttgtttaaattttagttttgtagaatattttcgttagtatttttggtttaatttttaattgtaagatttaatatttttatttaaggtttagtttaagtatttttcggtttttaaagtaaattatagagtagtaagattttaaatttgtttaaattttatttttgcgtggtagtttcgatttaaaataaagtaagttttagggaagtaaagtgataatgaaatttgtagtaggtttgttgattgaatagtgattgaattggtttgtgtttttaaggttagttattttagtttagtaattttttaagtacgtttaagatgtgtttgggaatataagaatttttgtcgagttattagtggatttttttgtttaatagtgtattatattatatagaaatattttggtcgggcgtggtgcgtttatatttataattttagtattttaggaggtttaggagggttgattatttgaggttaggagttttagattaatttgattaatatggtgaaatttaaaatttagtttttattaaaatttagtttttattaaaaatttaaaattagttatgtatggttgcgttatatttgtaattttagttatttgggaggttgaggtaggagaatagtttgaatttgagaggtggaggttgtagtgagtagagattaggttattgtattttagtttgggtaataagagtaaaattttgttttaaaaaaaaaagaaaaaagaaatagtttggtttagtttttttatttttttttcgggtgaaaaaaatggattgtttgtgatttaggagtgtcgggttttaaggtaaggaatatattatgttttttataagttgtttgttgaattaattggtggtatttttaggataagatagtttttaaagatagattgttagagtttaaatcgtggttttgttaattttgttggagataaaaagttttagtttttttatttataaaatggagataataatattattattaattagttattaaagagttattgtatatataagtgttagaatattgtttgttttagagtagtagtttttatttaatatttgtttttttattgaaggtgttagtaattagaaggtaattattttttttgttattgtgtgatacgtgaaagaatattagtttttaaataatttttaaaattggtaaggtatttagaatttgtgttttgtgaattattttattattagttaatgagattggataatttacgtagtaaagttttttatttttgtttaggagttttttttgggtattttatcgggggtgttgtttaggattttgataattggaggtttaatataggtaagatatttttttttataggttttatttattttttttgtatagttagaatttagaattgtttgattttttttttgtagtttttttggtttgtgattttttttttttattttttatttattttttttggttttttatagtttgtttgttaataaaatttgagttacggttgggtgtagtggtttacgtttataattttagtattttgggaggtcgaggtaggtagattatatggttaggagatcgagattattttggttaatatgatgaaatttcgtttttattaaaaatataaaaaattatttgggtatggtggtatatatttgtagttttagttattcgggaggttgaggtaggagaatcgtttgaatttagaaggcggaggttgtagtgatttaagatcgtgttattgtattttagtttggatgatagagtaagattttgttttaaaaaaaaaaaaaaaaaattttagttaataataatttacgttattaataaattgagatttttattagttattttgaattttttgttgagatatatgg

SEQ ID NO: 3 - DNA, 3397 bp, artificial sequence
  Description of the artificial sequence chemically pretreated human genomic DNA
ccaccattactctacccatatcccctccgttcgatacgcgacgcgtctaacaaaatactaaatacttaaaaaaccgctaactccgaacgaacgcaaacaaaataaaaaacgcgacgaaccgacgaaaccttatataccaaaaaaaccacaataaccgtaacgtaacgccgcccgcccaccccgactcccctcgccaaccgcccgccgcctcccgcccgcgtcgattccccaacgccccccgccgccgccgccaccgccaccgccgctctcgtcgcgaaccgataaaccccccgaacgacgcgccgctacaaataaacgaacgccgccgactactaataccgactatatcccgcccgactacgaactacgaacaaactaacacaaacccaacgtccgacacaaaacccgaaacacacacgcgaaccacacacgcatcgaatcgctaaaaaccgaacccaaatttcctttatttcccgaaacctaccatcttaccccccaaaaaaaaaatcgcgcctcgcgctacgaccgaaataaccgccgatcctaaaacaaatataaacgaacgaaactacaaaataatcgccccgaaaacccgaaaaaaactacgaattccaatacgaccgcgtaaaaaacgaacgccgaaacccctcgaacaaaattatctctcgacccgccgcaaaaaaaaaaaaaacgcgcgacgtaacgcgaacgcgaaccgaacgctcaccctaaccgacgaaacccgaacgccgcctaacgcgaacccttcccgactaatcgacgctcctcgcaaacgatatccgaatccgaaacgatctacgcgctaaccccgcgaccgcgccctccccgaaacccttactttattctataaacgcctcgtatcaaccaaacgcaataaactcacgaaaacgtcccgaatccgcatcttcccaaaaactaaaaaaccgctcgctaaacgcaaacccgctacctaacgctacgaaactacaaaatttcgatcccccgcgctcctacctacgacgaacgaaaaaaaaacgacccaaccctaaaaaaaccccctccgcgaccctaaaacccccaccgccctccccgaacgactctcctcccaaattctactcgaaaacaaaaacctcccctcctaacgatcccactacaattcaccgtccctctcgaattcctaaaaatacactttttcgatctctattacccgtcattcgtttaaaaaacaaaaccgattcgaaataaaaacatttacttatccgtaattaccattaaacaatttaataaaccgacacaaaaaaaaaattcgtcatcactctttaaattttaataaaatacttttttcctatccatcttattataaaaactttcaaacatacaacaaaaattaaaaataattttactaataaaatattttaaatttaaattttaattacaaaacttaatctttttattaaaaatttaacccaaatatttttcaatctttaaaacaaacaacaaaaaccaaacctaatataattataaatttatttaaatttcaattttacaaaacactctcgttaatatttttaattcaatttttaattacaaaacttaatacttttatttaaaatttaacccaaacatttttcgatctttaaaataaaccacaaaataataaaattctaaatttatctaaatttcacttttacgtaacaatctcgatttaaaacaaaataaatttcaaaaaaacaaaataacaataaaatctacaataaatttattaactaaataataactaaattaatttataccttcaaaatcaatcactttaactcaataactttccaaacacgtttaaaatatacttaaaaacacaaaaatttctatcgaaccaccaataaactttcctatttaacaatataccacattatataaaaatactttaaccgaacgtaatacgctcacacctataatcccaacactttaaaaaacttaaaaaaactaatcacctaaaatcaaaaattccaaaccaacctaaccaacataataaaacctaaaatctaatctctactaaaatttaatctctactaaaaatccaaaattaaccatacataattacgtcatacctataatcccaactacttaaaaaactaaaacaaaaaaataacttaaacctaaaaaataaaaattacaataaacaaaaatcaaaccattacattccaacctaaacaacaaaaacaaaactctatctcaaaaaaaaaaaaaaaaaaaaataatctaacccaacttctccattccttttccgaataaaaaaaataaactatttataactcaaaaataccgaattccaaaacaaaaaacatactatatcctccacaaactacttattaaactaactaataatattcctaaaataaaacaacctctaaaaacaaactaccaaaattcaaaccgtaactctactaattttactaaaaacaaaaaacctcaattccttcatctataaaataaaaataataacatcattactaattaattattaaaaaattattatatatataaatattaaaatactacctacctcaaaataataactctcacttaatatttactcttccactaaaaataccaacaaccaaaaaataattaccttctctaccactatataatacgtaaaaaaatatcaatctccaaataattcccaaaactaacaaaacactcaaaatctatactttataaaccatttcatcaccaactaataaaactaaataattcacgtaacaaaacccttcacctctacccaaaaattccccctaaacatcctaccgaaaatattacccaaaactttaacaactaaaaacccaacacaaataaaatacttctctttacaaatctcacctaccttttctatacaactaaaactcaaaactacctaatttttcctttacaattcttttaacttataattctttcttcctattctttatccacttctcctaatttttcataatttatttactaataaaacctaaaccacgactaaatacaataactcacgcctataatcccaacactttaaaaaaccgaaacaaacaaatcacataatcaaaaaatcgaaaccatcctaactaacataataaaaccccgtctctactaaaaatacaaaaaattacctaaacataataacacacacctataatcccaactactcgaaaaactaaaacaaaaaaatcgcttaaacccaaaaaacgaaaattacaataacccaaaatcgtaccactacactccaacctaaataacaaaacaaaactctatctcaaaaaaaaaaaaaaaaaaccttaaccaataataactcacgccactaacaaattaaaactcttaccaattatcctaaactttttattaaaacacataa

SEQ ID NO: 4 - DNA, 3397 bp, artificial sequence
  Description of the artificial sequence chemically pretreated human genomic DNA
ccatatatctcaacaaaaaattcaaaataactaataaaaatctcaatttattaataacgtaaattattattaactaaaattttttttttttttttttaaaacaaaatcttactctatcatccaaactaaaatacaataacacgatcttaaatcactacaacctccgccttctaaattcaaacgattctcctacctcaacctcccgaataactaaaactacaaatatataccaccatacccaaataattttttatatttttaataaaaacgaaatttcatcatattaaccaaaataatctcgatctcctaaccatataatctacctacctcgacctcccaaaatactaaaattataaacgtaaaccactacacccaaccgtaactcaaattttattaacaaacaaactataaaaaaccaaaaaaaataaataaaaaataaaaaaaaaaaatcacaaaccaaaaaaactacaaaaaaaaaatcaaacaattctaaattctaactatacaaaaaaaataaataaaacctataaaaaaaaatatcttacctatattaaacctccaattatcaaaatcctaaacaacacccccgataaaatacccaaaaaaaactcctaaacaaaaataaaaaactttactacgtaaattatccaatctcattaactaataataaaataattcacaaaacacaaattctaaataccttaccaattttaaaaattatttaaaaactaatattctttcacgtatcacacaataacaaaaaaaataattaccttctaattactaacaccttcaataaaaaaacaaatattaaataaaaactactactctaaaacaaacaatattctaacacttatatatacaataactctttaataactaattaataataatattattatctccattttataaataaaaaaactaaaactttttatctccaacaaaattaacaaaaccacgatttaaactctaacaatctatctttaaaaactatcttatcctaaaaataccaccaattaattcaacaaacaacttataaaaaacataatatattccttaccttaaaacccgacactcctaaatcacaaacaatccatttttttcacccgaaaaaaaaataaaaaaactaaaccaaactatttcttttttcttttttttttaaaacaaaattttactcttattacccaaactaaaatacaataacctaatctctactcactacaacctccacctctcaaattcaaactattctcctacctcaacctcccaaataactaaaattacaaatataacgcaaccatacataactaattttaaatttttaataaaaactaaattttaataaaaactaaattttaaatttcaccatattaatcaaattaatctaaaactcctaacctcaaataatcaaccctcctaaacctcctaaaatactaaaattataaatataaacgcaccacgcccgaccaaaatatttctatataatataatacactattaaacaaaaaaatccactaataactcgacaaaaattcttatattcccaaacacatcttaaacgtacttaaaaaattactaaactaaaataactaaccttaaaaacacaaaccaattcaatcactattcaatcaacaaacctactacaaatttcattatcactttacttccctaaaacttactttattttaaatcgaaactaccacgcaaaaataaaatttaaacaaatttaaaatcttactactctataatttactttaaaaaccgaaaaatacttaaactaaaccttaaataaaaatattaaatcttacaattaaaaattaaaccaaaaatactaacgaaaatattctacaaaactaaaatttaaacaaatttacaaccatactaaatttaatctctactatttactttaaaaactaaaaaacacttaaactaaaccttcaataaaaaaattaaatcttacaattaaaatttaaactcaaaatactccaccaataaaattactttcaacttttattatatacttaaaaattttcacaataaaataaataaaaaaaaaatattccaccaaaatctaaaaaataataacgaatctcctctttataccgattcattaaattacctaataacaactacgaataaataaatacctttatctcgaaccgattctactctttaaacgaataacgaacaacaaaaaccgaaaaaatatatccccaaaaattcgaaaaaaacgataaattataataaaaccgccaaaaaaaaaaattcctacccccgaataaaacctaaaaaaaaaaccgctcgaaaaaaacgataaaaacttcaaaaccgcgaaaaaaatcccctcaaaattaaaccgtctcctcctcgcccgccgcaaacaaaaacgcgaaaaaccgaaaccctataatttcgcaacgtcaaacaacgaatctacgcccaacgaacgactccccaactcctaaaaaaatacgaacccgaaacgcccccgtaaactcactacgcctaactaacacgaaacgctcacaaaacaaaacaaaaacttcgaaaaaaacgcgaccgcgaaaccaacgcgcaaatcgctccgaacccgaacaccgcctacgaaaaacgccgaccaaccgaaaaaaattcgcgctaaacgacgcccgaatcccgtcgaccaaaataaacgtccgacccgcgtccgcgccacgccgcgcgttcccctttcctctacgacgaaccgaaaaataaccctacccgaaaaatcccgacgcccgccccccacgcgatcgcactaaaattcgcaacccctctcgaatccccgaaacgatcattttacaatctcgctcgcccacacttactccaaaaccgacgactaccccgaccgcaacgcgaaacgcgacttttcttttaaaaaataaaataacaaatcccgaaaaacaaaaaaaacttaaacccgacccccaacgatccgatacgtatataactcgcgtatataccccgaatcctataccgaacgctaaacctataccaacctacccgcaacccgcaaccgaacgaaatacaaccgataccaacaaccgacgacgcccgcccacctacaacgacgcgccgcccgaaaaactcaccgacccgcgacgaaaacgacgataacgataacgacgacgacgaaaaacgctaaaaaaccgacgcgaacgaaaaacgacgaacgactaacgaaaaaaaccgaaataaacgaacgacgccacgtcacgactattataactttcctaatatataaaatctcgccgactcgccgcgctccccaccttacctacgcccgcccgaaaccaacgattctccaaacacccaacatcctactaaacgcgccgcgcaccgaacgaaaaaaacataaacaaaacaataataa

SEQ ID NO: 5 - DNA, 18 bp, artificial sequence
  Description of the artificial sequence chemically pretreated human genomic DNA
gggtaagatggtaggttt

SEQ ID NO: 6 - DNA, 19 bp, artificial sequence
  Description of the artificial sequence chemically pretreated human genomic DNA
cattactctacccatatcc

SEQ ID NO: 7 - DNA, 13 bp, artificial sequence
  Description of the artificial sequence chemically pretreated human genomic DNA
tttgttcgtagtt

(19) United States
(12) Patent Application Publication
(10) Pub. No.: US 2004/0091881 A1
(43) Pub. Date: May 13, 2004

(54) Diagnosis of diseases which are associated with cd24

(76) Inventors: Alexander Olek, Berlin (DE); Christian Piepenbrock, Berlin (DE); Kurt Berlin, Stahnsdorf (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/343,502

(22) PCT Filed: Aug. 2, 2001
(86) PCT No.: PCT/EP01/08969

(30) Foreign Application Priority Data
Aug. 3, 2000 (DE) 100377696

(51) Int. Cl. C12Q 1/68; C07H 21/04; C07K 9/00

(52) U.S. Cl. 435/6; 530/322; 536/24.3; 530/324

(57) ABSTRACT

The invention relates to the chemically modified genomic sequence of the CD24 gene, to oligonucleotides oriented against the sequence and/or PNA oligomers for detecting the cytosine methylation status of the CD24 gene and to a method for determining genetic and/or epigenetic parameters of the CD24 gene.

The present invention relates to nucleic acids, oligonucleotides, PNA-oligomers and to a method for the diagnosis and/or therapy of diseases which have a connection with the genetic and/or epigenetic parameters of the cell surface antigen CD24 and, in particular, with the methylation status thereof.

The levels of observation that have been well studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

The human cell surface antigen CD24 is a glycosyl-phophatidyl inositole (GPI)—coupled glycoprotein. GPI-coupled proteins are involved in the signal transduction that is mediated by members of the protein tyrosine kinase family. CD24 is involved in the differentiation and activation of granulocytes and B-lymphocytes. Changes in the expression of CD24 take place at critical times during the development of B-stem cells. CD24 is localised on the human chromosome 6p21.

In contrast to the expression in many B-stem cells as well as mature granulocytes, experiments with monoclonal antibodies indicate that most other hematopoietic cells, including T-cells, monocytes, red blood cells, and thrombocytes do not express the CD24 antigen.

In addition to the association of CD24 with different diseases, it can also be used as marker for diseases (Tsutsudaasano A., Migita M., Takahashi K, Shimada T. Transduction of fibroblasts and CD34+ progenitors using a selectable retroviral vector containing cDNAs encoding arylsulfatase A and CD24 J Hum Genet. 2000; 45(1):1823.). Thus, CD24 is used as marker for human breast carcinoma and possibly supports metastasis during the interaction between tumour cells and thrombocytes or endothelial cells (Fogel M, Friederichs J, Zeller Y, Husar M, Smirnov A, Roitman L, Altevogt P, Sthoeger Z M. CD24 is a marker for human breast carcinoma. Cancer Lett. Aug. 23, 1999;143(1):87-94.).

CD24 is also expressed on nasophryngeal carcinoma cells (Karran L, Jones M, Morley G, van Noorden S, Smith P, Lampert I, Griffin BE. Expression of a B-cell marker, CD24, on nasopharyngeal carcinoma cells. Int J Cancer. Feb. 8, 1995;60(4):562-6.).

Furthermore, CD24 can also be used in the diagnosis for splenic lymphoma with villous lymphocytes (SLVL) (Troussard X, Valensi F, Duchayne E, Garand R, Felman P, Tulliez M, Henry-Amar M, Bryon P A, Flandrin G. Splenic lymphoma with villous lymphocytes: clinical presentation, biology and prognostic factors in a series of 100 patients. Groupe Francais d'Hematologie Cellulaire. Br J Haematol. 1996). In connection with modified cellular phenotypes (amongst others, CD24), SLVL could be diagnosed in one patient (Kuwayama M, Machii T, Yamaguchi M, Yamaguti K, Kitani T, Kanakura Y. Blastic transformation of splenic lymphoma with villous lymphocytes after a well-controlled chronic phase of more than 10 years. Int J Hematol. February 2000;71(2):167-71).

The infantile spinal muscle atrophy exhibits an abnormal expression patterns of cell surface proteins, such as CD24 (Soubrouillard C, Pellissier J F, Lepidi H, Mancini J, Rougon G, Figarella-Branger D. Expression of developmentally regulated cytoskeleton and cell surface proteins in childhood spinal muscular atrophies. J Neurol Sci. November 1995;133(1-2):155-63). CD24 is over-expressed in lung cancers. It is believed that the CD24 promoter has a strong cell-type specific activity (Pass M K, Quintini G, Zarn J A, Zimmermann S M, Sigrist J A, Stahel R A. The 5′-flanking region of human CD24 gene has cell-type-specific promoter activity in small-cell lung cancer. Int J Cancer. Nov. 9, 1998;78(4):496-502).

CD24 monoclonal antibodies can be used in the diagnosis of the Epstein-Barr Virus induced lymphoproliferative syndrome (EBV-LPS) (Lazarovits A I, Tibbles L A, Grant D R, Ghent C N, Wall W J, White M J, Joncas J H. Anti-B cell antibodies for the treatment of monoclonal Epstein-Barr virus-induced lymphoproliferative syndrome after multivisceral transplantation. Clin Invest Med. December 1994;17(6):621-5).

The involvement of CD24 associated complexes in lung cancers and leukemias is discussed (Zarn J A, Zimmermann S M, Pass M K, Waibel R, Stahel R A. Association of CD24 with the kinase c-fgr in a small cell lung cancer cell line and with the kinase lyn in an erythroleukemia cell line. Biochem Biophys Res Commun. Aug. 14, 1996;225(2):384-91).

The involvement of CD24 in acute lymphoblast leukemia is supported by the fact that with patients having this disease a low CD24/CD45 antigen density is associated with a positive indication for the patient (Lavabre-Bertrand T, Duperray C, Brunet C, Poncelet P, Exbrayat C, Bourquard P, Lavabre-Bertrand C, Brochier J, Navarro M, Janossy G. Quantification of CD24 and CD45 antigens in parallel allows a precise determination of B-cell maturation stages: relevance for the study of B-cell neoplasias. Leukemia. March 1994;8(3):402-8).

An increased expression of CD24 was found in patients with rheumatic and reactive arthritis (Felzmann T, Gadd S, Majdic O, Maurer D, Petera P, Smolen J, Knapp W. Analysis of function-associated receptor molecules on peripheral blood and synovial fluid granulocytes from patients with rheumatoid and reactive arthritis. J Clin Immunol. July 1991;11(4):205-12). Furthermore, a connection between CD24 and multiple myeloma (Duperray C, Bataille R, Boiron J M, Haagen I A, Cantaloube J F, Zhang X G, Boucheix C, Klein B. No expansion of the pre-B and B-cell compartments in the bone marrow of patients with multiple myeloma. Cancer Res. Jun. 15, 1991;51(12):3224-8) and between CD24 and Waldenstrom's macroglobulinemia (Jensen G S, Andrews E J, Mant M J, Vergidis R, Ledbetter J A, Pilarski L M. Transitions in CD45 isoform expression indicate continuous differentiation of a monoclonal CD5+ CD11b+ B lineage in Waldenstrom's macroglobulinemia. Am J Hematol. May 1991;37(1):20-30) could be shown.

Important diseases are described as follows:

multiple myeloma:

i. Approximately three thousand humans die from mulitple myeloma (or plasmacytoma) per year. Currently, there is no cure for the disease. It is a matter of a systematic disease with a neoplastic increase of plasma cells and formation of paraproteins with increased total protein as well as a strong increase of the beta-to gamma-globulin fraction in most cases. In multiple myeloma, plasma cells grow uncontrolled. These cells are normally formed as “agents” of the immune system in bone marrow and introduced into the blood circulation. There, they search for disease-causing agents, poisons and cancerous cells. By a genetic defect, they themselves can mutate into tumour cells. A consequence of this is a weakening of the immune system, destruction of bone structure, anaemia and severe pain in the patients.

reactive arthritis:

i. The reactive arthritis belongs to the infectious rheumatic diseases. The joints are increasingly damaged by the continuous infections. Commonly, this causes massive handicaps and pain. The disease is caused, for example, by bacteria, in particular by the so called borreliae or chlamydias. If the disease is chronic, the joints suffer from similar damages, as in rheumatoid arthritis. In both rheumatic forms, the cytokines formed by the immune cells importantly contribute to the destruction of joints. The immune cells of rheumatic patients increasingly produce the inflammation-supporting cytokine “interleukin 1” and the “tumour-necrosis-factor-alpha (TNF-alpha). It must be assured in which rheumatic disease which cytokine is increasingly produced, before one can specifically steer against the respective messenger compounds. In rheumatic arthritis, in particular T1-helper cells heat up the inflammation, in contrast to T2-helper cells in reactive arthritis.

Splenic-lymphoma with villous lymphocytes:

i. The splenic-lymphoma with villous lymphocytes (SLVL) is a chronic lymphoproliferative disease and is characterised by splenic megaly and the presence of atypical lymphocytes with villous protuberances in the peripheral blood and in bone marrow. SLVL mainly occurs in men from 70 years of age. In this rare disease, no chromosomal abnormalities are known; it is characterised by circulating lymphocytes with thin and short cytoplasmatic villi and an increase of the spleen, in two thirds of the cases a monoclonal gammopathy occurs. The cellular origin of the disease underlies somatic hypermutations, before the tumour transformation.

Macroglobulinemia Waldenstrom:

i. The macroglobulinemia Waldenstrom is a rare immune proliferative disease and is characterised by an increase of macroglobulines in serum. The histologic features of the bone marrow are regarded as prognostically relevant in related B-cell neoplasms, such as multiple myeloma and the chronic lymphatic leukemia. All patients with macroglobulinemia Waldenstrom exhibit a circulating tumour marker, the monoclonal IgM protein. In some cases, high levels of monoclonal IgM protein can produce a hyperviscosity syndrome, which is manifested by oronasal bleedings.

Epstein-Barr-Virus induced lymphoproliferative syndrome:

i. EBV is a ubiquitous herpes virus (herpes-subtype IV) with a high degree of infection rate (approximately 95% of adults) in the population. The EBV-associated symptom is commonly mononucleosis (=glandular fever) which mostly occurs in the adolescence (15-25 years of age) without many symptoms and leaves a life-long presence of the virus. After first infection, adults secrete EBV with the salvia in different amounts for their lifetime. EBV has a particular meaning in humans, who happen to enter an immune suppressive phase due to other diseases. Major symptoms of the EBV-infection (incubation time 4-6 weeks) are flue-like complaints such as headache, adynaemia and high fever, in a further course pharyngitis, splenic megaly and lymphadenitis. In severe courses, EBV-induced thrombopenia, hepatitis and encephalitis are described. EBV is furthermore also suspected to play a possible co-causal role in the development of the malignant lymphoma, in particular since T- and in particular B-lymphocytes account for the target cells of the virus. In cases of a chronic EBV-infection, the risk for T- and B-lymphoma is clearly increased. Also several lymphoproliferative diseases, such the Gullian-Barre- and the Budd-Chiari-syndrome are counted among the EBV-associated symptoms.

infantile spinal muscle atrophy:

i. The spinal muscle atrophy is an inherited degeneration of the cells of the motoric anterior horn and cerebral nerve cores of the brain stem which leads to a limp purely motoric paralysis. The outer appearance is very similar to the one of muscle dystrophy. One form, the infantile spinal muscle atrophy, is not gender-specific, probably recessively inheritable, occurs in the age between 0-2 months, and progresses rapidly. In addition, there is a chronic form which occurs in the age of 0-2 years and progresses slower.

acute lymphatic leukaemia:

i. Commonly, a leukaemia is understood as the malignant degeneration and distortion of the maturation of white blood cells. In most cases, a strong increase of immature white blood cells occurs, which gradually displaces the regular white blood cells. The results are anaemia, bleeding, infections and distortions of the organ functions. The basis for this disease is, as in most cancerous diseases, unknown. If mainly immature lymphocytes are present, it is spoken of lymphatic leukaemia. The acute lymphatic leukaemia (ALL) is the most frequent form of leukaemia and also the most frequent type of cancer in children. In the fast progressing ALL, the maturating lymphocytes become too numerous and do not fully mature. The precursor cells of the lymphocytes from the bone marrow transform cancerously and can be found in the blood and bone marrow. The cancerous-like lymphocytes disperse in the body via the blood circulation, for example, to the liver, spleen, spinal marrow or in the cerebrum.

lung cancer (small cell bronchial carcinoma):

i. Lung cancer represents the most frequent malignant disease worldwide. Small cell bronchial carcinomas constitute 25-30% of all lung cancers. This is a matter of a disease, in which malignant cancer cells can be found in lung tissue. It is characterised by a particularly high and fast proliferation. The small cell bronchial carcinoma clinically behaves very malignant and leads to an early metastasis. In 80% of the patients metastases can already be found during the first diagnosis. Some of these tumours can secrete hormones into the blood and thereby affect the natural housekeeping of hormones.

nasopharyngeal carcinoma:

i. The nasopharyngeal carcinoma (or lymphoepithelioma) is a malignant tumour of lymphoepithelial organs and consists of malignant epithelial cells. It is the most commonly occurring malignant tumour of the nasopharyngeal region and is characterised by its rapid growth. Furthermore, it is characterised by an early metastasis and high radiation sensitivity. It is endemic for some regions and also occurs decades after the first infection with Epstein-Barr-Virus.

5-Methylcytosine is the most frequent covalent base modification in the DNA of eukaryotic cells. It plays a role, for example, in the regulation of the transcription, in genetic imprinting, and in tumourigenesis. Therefore, the identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification.

A relatively new and currently the most frequently used method for analysing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. In terms of sensitivity, the prior art is defined by a method which encloses the DNA to be analysed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. Dec. 15, 1996;24(24):5064-6). Using this method, it is possible to analyse individual cells, which illustrates the potential of the method. However, currently only individual regions of a length of up to approximately 3000 base pairs are analysed, a global analysis of cells for thousands of possible methylation events is not possible. However, this method cannot reliably analyse very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. March-April 1997;5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. November 1997;17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. Jun. 15, 1997;25(12):2529-31, WO-Patent 9500669) or by enzymatic digestion (Xiong Z, Laird P W. COBRA: a sensitive and quantitative DNA methylation assay. Nucleic Acids Res. Jun. 15, 1997;25(12):2532-4). In addition, detection by hybridisation has also been described (Olek et al., WO 99 28498).

Further publications related to the use of the bisulfite technique for methylation detection in individual genes are: Grigg G, Clark S. Sequencing 5-methylcytosine residues in genomic DNA. Bioessays. June 1994;16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. March 1997;6(3):387-95; Feil R, Charlton J, Bird A P, Walter J, Reik W. Methylation analysis on individual chromosomes: improved protocol for bisulphite genomic sequencing. Nucleic Acids Res. Feb. 25, 1994;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. May 19, 1995;157(1-2):261-4; WO 97 46705, WO 95 15373 and WO 45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionisation of proteins with molecular masses exceeding 10,000 daltons. Anal Chem. Oct. 15, 1988;60(20):2299-301). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. Apr. 25, 1995;23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

In view of the above, it is the object of the present invention to provide the chemically modified DNA of the gene CD24, as well as oligonucleotides and/or PNA-oligomers for detecting cytosine methylations, as well as a method which is particularly suitable for the diagnosis and/or therapy of genetic and epigenetic parameters of the gene CD24. The present invention is based on the discovery that genetic and epigenetic parameters and, in particular, the cytosine methylation pattern of the gene CD24 are particularly suitable for the diagnosis and/or therapy of diseases associated with CD24.

This objective is achieved according to the present invention by providing a nucleic acid comprising a sequence at least 18 bases in length of a segment of the chemically pretreated DNA of the gene of CD24 according to one of the Seq. ID No.1 to Seq. ID No.4. Surprisingly, it could be found that the chemically modified nucleic acid could heretofore not be connected with the ascertainment of genetic and epigenetic parameters.

The object of the present invention is further achieved by an oligonucleotide or PNA-oligomer for the detection of the cytosine methylation status in chemically modified DNA comprising at least one base sequence having a length of at least 9 nucleotides which hybridises to a chemically pretreated DNA of the gene of CD24 according to one of the Seq. ID No.1 to Seq. ID No.4. The oligomer probes according to the present invention constitute important and effective tools which, for the first time, make it possible to ascertain the genetic and epigenetic parameters of CD24. The base sequence of the oligomers preferably contains at least one CpG dinucleotide. The probes may also exist in the form of a PNA (peptide nucleic acid) which has particularly preferred pairing properties. Particularly preferred are oligonucleotides according to the present invention in which the cytosine of the CpG dinucleotide is located approximately in the middle third of the oligomer, for example in which the cytosine of the CpG dinucleotide is the 5^{th}-9^{th} nucleotide from the 5′-end of a, e.g., 13-mer. In the case of PNA-oligomers, it is preferred for the cytosine of the CpG dinucleotide to be the 4^{th}-6^{th} nucleotide from the 5′-end of a, e.g., 9-mer.

The oligomers according to the present invention are normally used in so called “sets” which contain at least one oligomer for at least one of the CpG dinucleotides of one of the sequences according to Seq. ID No.1 to Seq. ID No.4. Preferred is a set which comprises at least one oligomer for each of the CpG dinucleotides of one of Seq. ID No.1 to Seq. ID No.4.

Moreover, the present invention makes available a set of at least two oligonucleotides which can be used as so-called “primer oligonucleotides” for amplifying DNA sequences of one of Seq. ID No.1 to Seq. ID No.4 or segments thereof.

In the case of the sets of oligonucleotides according to the present invention, it is preferred that at least one oligonucleotide is bound to a solid phase.

The present invention moreover relates to a set of at least 10 (oligonucleotides and/or PNA-oligomers) used for detecting the cytosine methylation state in chemically pretreated genomic DNA (Seq. ID No.1 to Seq. ID No.4). These probes enable diagnosis and/or therapy of genetic and epigenetic parameters of the gene CD24. The set of oligomers may also be used for detecting single nucleotide polymorphisms (SNPs) in the chemically pretreated DNA of the gene CD24 according to one of Seq. ID No.1 to Seq. ID No.4.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices are possible as well.

Therefore, a further subject matter of the present invention is a method for manufacturing an array fixed to a carrier material for analysis in connection with diseases associated with CD24, in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the analysis of diseases associated with CD24 which contains at least one nucleic acid according to the present invention. DNA chips are known, for example, from U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond or are complementary to an 18 base long segment of the base sequences specified in the appendix (Seq. ID No.1 to Seq. ID No.4), oligonucleotides and/or PNA-oligomers as well as instructions for carrying out and evaluating the described method. However, a kit along the lines of the present invention can also contain only part of the aforementioned components.

The present invention furthermore relates to a method for producing a diagnostic agent for the diagnosis of diseases associated with CD24 by analysing the methylation patterns of the gene CD24, wherein the diagnostic agent is characterised in that at least one nucleic acid according to the present invention is used for its production, optionally together with suitable auxiliary compounds and additives.

Moreover, the present invention relates to a diagnostic agent for the diagnosis of diseases associated with CD24 by analysing the methylation patterns of the gene CD24, wherein the diagnostic agent comprises at least one nucleic acid according to the present invention, optionally together with suitable auxiliary compounds and additives.

The present invention also provides a method for ascertaining genetic and/or epigenetic parameters of the gene CD24 by analysing cytosine methylations and single nucleotide polymorphisms, comprising the following steps:

In the first step of the method, a genomic DNA sample is chemically treated in such a manner that cytosine bases which are unmethylated at the 5′-position are converted to uracil, thymine, or another base which is dissimilar to cytosine in terms of hybridisation behaviour. This will be understood as “chemical pretreatment” hereinafter.

The genomic DNA to be analysed is preferably obtained form usual sources of DNA such as cells or cell components, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, or combinations thereof.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behaviour.

Fragments of this chemically pretreated DNA are amplified, using sets of primer oligonucleotides according to the present invention, and a, preferably, heat-stable polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

In a preferred embodiment of the method, the set of primer oligonucleotides includes at least two olignonucleotides whose sequences are each reverse complementary or identical to an at least 18 base-pair long segment of the base sequences specified in the appendix (Seq. ID No.1 to Seq. ID No.4). The primer oligonucleotides are preferably characterised in that they do not contain any CpG dinucleotide.

According to the present invention, it is preferred that at least one primer oligonucleotide is bonded to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminium, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualised by means of matrix assisted laser desorption/ionisation mass spectrometry (MALDI) or using electron spray mass spectrometry (ESI).

The amplificates obtained in the second step of the method are subsequently hybridised to an array or a set of oligonucleotides and/or PNA probes. In this context, the hybridisation takes place in the manner described in the following. The set of probes used during the hybridisation is preferably composed of at least 10 oligonucleotides or PNA-oligomers. In the process, the amplificates serve as probes which hybridise to oligonucleotides previously bonded to a solid phase. The non-hybridised fragments are subsequently removed. Said oligonucleotides contain at least one base sequence having a length of 13 nucleotides which is reverse complementary or identical to a segment of the base sequences specified in the appendix, the segment containing at least one CpG dinucleotide. The cytosine of the CpG dinucleotide is the 5^{th} to 9^{th} nucleotide seen from the 5′-end of the 13-mer. One oligonucleotide is present for each CpG dinucleotide. Said PNA-oligomers comprise at least one base sequence having a length of 9 nucleotides which is reverse complementary or identical to a segment of the base sequences specified in the appendix, the segment containing at least one CpG dinucleotide. The cytosine of the CpG dinucleotide is the 4^{th} to 6^{th} nucleotide seen from the 5′-end of the 9-mer. One oligonucleotide is present for each CpG dinucleotide.

In the fourth step of the method, the non-hybridised amplificates are removed.

In the final step of the method, the hybridised amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualised by means of matrix assisted laser desorption/ionisation mass spectrometry (MALDI) or using electron spray mass spectrometry (ESI).

The produced fragments may have a single positive or negative net charge for better detectability in the mass spectrometer. The aforementioned method is preferably used for ascertaining genetic and/or epigenetic parameters of the gene CD24.

The oligomers according to the present invention or arrays thereof as well as a kit according to the present invention are intended to be used for the diagnosis of a disease associated with the gene CD24 by analysing methylation patterns of the gene CD24. According to the present invention, the method is preferably used for the diagnosis of important genetic and/or epigenetic parameters within the gene CD24.

The method according to the present invention is used, for example, for the diagnosis of cancerous diseases, for example leukaemia, lung cancer or the nasopharyngeal karcinoma, multiple myeloma, reactive arthritis, spleen-lymphoma, Waldenstrom macroglobulinemia, Epstein-Barr-Virus induced syndrome and/or infantile spinal muscle atrophy.

The nucleic acids according to the present invention of Seq. ID No.1 to Seq. ID No.4 can also be used for the diagnosis of genetic and/or epigenetic parameters of the gene CD24. Moreover, the oligomers according to the invention or an arrangement thereof a kit can be used for the diagnosis of a disease associated with CD24 by analysing the methylation patterns of the gene CD24. The analysis is performed according to the above methods and mentioned in the examples. All diseases concurrent with the change of the methylation patterns of the gene CD24 can be diagnosed, for example cancerous diseases, for example leukaemia, lung cancer or the nasopharyngeal karcinoma, multiple myeloma, reactive arthritis, spleen-lymphoma, Waldenstrom macroglobulinemia, Epstein-Barr-Virus induced syndrome and/or infantile spinal muscle atrophy.

The present invention moreover relates to the diagnosis and/or prognosis of events which are disadvantageous to patients or individuals in which said disadvantageous events are associated with methylation patterns of the gene CD24. Based on the data obtained by means of the present invention with respect to the changes of the methylation pattern of the patient, the examining person can perform the diagnosis and/or prognosis of events which are disadvantageous for the patient. For this, the data obtained by means of a method according to the present invention can be used. For example, the important genetic and/or epigenetic parameters within the gene CD24, said parameters obtained by means of the present invention may be compared to another set of genetic and/or epigenetic parameters, the differences serving as the basis for a diagnosis and/or prognosis of events which are disadvantageous to patients or individuals.

In the context of the present invention the term “hybridisation” is to be understood as a bond of an oligonucleotide to a completely complementary sequence along the lines of the Watson-Crick base pairings in the sample DNA, forming a duplex structure. To be understood by “stringent hybridisation conditions” are those conditions in which a hybridisation is carried out at 60° C. in 2.5×SSC buffer, followed by several washing steps at 37° C. in a low buffer concentration, and remains stable.

The term “functional variants” denotes all DNA sequences which are complementary to a DNA sequence, and which hybridise to the reference sequence under stringent conditions and have an activity similar to the corresponding polypeptide according to the present invention.

In the context of the present invention, “genetic parameters” are mutations and polymorphisms of the gene CD24 and sequences further required for its regulation. To be designated as mutations are, in particular, insertions, deletions, point mutations, inversions and polymorphisms and, particularly preferred, SNPs (single nucleotide polymorphisms). Nevertheless, polymorphisms can also be insertions, deletions or inversions.

In the context of the present invention, “epigenetic parameters” are, in particular, cytosine methylations and further chemical modifications of DNA bases of the gene CD24 and sequences further required for its regulation. Further epigenetic parameters include, for example, the acetylation of histones which, however, cannot be directly analysed using the described method but which, in turn, correlates with the DNA methylation.

In the following, the present invention will be explained in greater detail on the basis of the sequences and examples and the accompanying figure without being limited thereto.

Seq. ID No. 1 shows the sequence of the chemically pretreated genomic DNA of the gene CD24.

Seq. ID No. 2 shows the sequence of a second chemically pretreated genomic DNA of the gene CD24.

Seq. ID No. 3 shows the reverse complementary sequence of Seq. ID No. 1 of chemically pretreated genomic DNA of the gene CD24.

Seq. ID No. 4 shows the reverse complementary sequence of Seq. ID No. 2 of chemically pretreated genomic DNA of the gene CD24.

Seq. ID No. 5 shows the sequence of an oligonucleotide for the amplification of CD24 from Example 1.

Seq. ID No. 6 shows the sequence of a second oligonucleotide for the amplification of CD24 from Example 1.

Seq. ID No. 7 shows the sequence of an oligonucleotide for hybridising of the amplificate of CD24 from Example 1.

The following example relates to a fragment of the gene CD24 in which a specific CG-position is analysed for its methylation status.

### EXAMPLE 1

### Accomplishment of the Methylation Analysis in the Gene CD24

In the first step, a genomic sequence is treated using bisulfite (hydrogen sulfite, disulfite) in such a manner that all cytosines which are not methylated at the 5-position of the base are modified in such a manner that a different base is substituted with regard to the base pairing behaviour, while the cytosines methylated at the 5-position remain unchanged. If bisulfite solution in a concentration range between 0.1 and 6M is used for the reaction, then an addition takes place at the non-methylated cytosine bases. Moreover, a denaturating reagent or solvent as well as a radical interceptor is present. A subsequent alkaline hydrolysis then gives rise to the conversion of non-methylated cytosine nucleobases to uracil. Chemically converted DNA is then used for the detection of methylated cytosines. In the second method step, the treated DNA sample is diluted with water or an aqueous solution. Preferably, the DNA is subsequently desulfonated (10-30 min, 90-100° C.) at an alkaline pH value. In the third step of the method, the DNA sample is amplified in a polymerase chain reaction, preferably using a heat-resistant DNA polymerase. In the present case, cytosines of the gene CD24, here from the promoter region, are analysed. To this end, a defined fragment having a length of 489 bp is amplified with the specific primer oligonucleotides GGGTAAGATGGTAGGTTT (Seq. ID No. 5) and CATTACTCTACCCATATCC (Seq. ID No. 6). This amplificate serves as a sample which hybridises to an oligonucleotide previously bonded to a solid phase, forming a duplex structure, for example TTTGTTCGTAGTT (Seq. ID No. 7), the cytosine to be detected being located at position 125 of the amplificate. The detection of the hybridisation product is based on Cy3 and Cy5 flourescently labelled primer oligonucleotides which have been used for the amplification. The hybridisation reaction of the amplified DNA with the oligonucleotide takes place only if a methylated cytosine was present at this location in the bisulfite treated DNA. Thus, the methylation status of the specific cytosine to be analysed may be inferred from the hybridisation product.

### EXAMPLE 2

### Diagnosis of Diseases Associated with CD24

In order to relate the methylation patterns to one of the diseases associated with CD24, it is initially required to analyze the DNA methylation patterns of a group of diseased and of a group of healthy patients. These analyses are carried out, for example, analogously to example 1. The results obtained in this manner are stored in a database and the CpG dinucleotides which are methylated differently between the two groups are identified. This can be carried out by determining individual CpG methylation rates as can be done, for example, in a relatively imprecise manner, by sequencing or else, in a very precise manner, by a methylation-sensitive “primer extension reaction”. It is also possible for the entire methylation status to be analyzed simultaneously, and for the patterns to be compared, for example, by clustering analyses which can be carried out, for example, by a computer.

Subsequently, it is possible to allocate the examined patients to a specific therapy group and to treat these patients selectively with an individualised therapy.

Example 2 can be carried out, for example, for the following diseases: multiple myeloma, reactive arthritis, spleen-lymphoma, Waldenstrom macroglobulinemia, Epstein-Barr-Virus induced syndrome, infantile spinal muscle atrophy, leukaemia, lung cancer and nasopharyngeal karcinoma.

1. A nucleic acid comprising a sequence at least 18 bases in length of a segment of the chemically pretreated DNA of the gene of CD24 according to one of the Seq. ID No.1 to Seq. ID No.4.

2. An oligomer (oligonucleotide or PNA-oligomer) for the detection of the cytosine methylation status in chemically modified DNA comprising at least one base sequence having a length of at least 9 nucleotides which hybridises to a chemically pretreated DNA of the gene of CD24 according to one of the Seq. ID No.1 to Seq. ID No.4.

3. The oligomer as recited in claim 2, wherein the base sequence comprises at least one CpG dinucleotide.

4. The oligomer as recited in claim 2 or 3 in form of a PNA (peptide nucleic acid).

5. The oligomer as recited in claim 3, characterised in that the cytosine of the CpG dinucleotide is located approximately in the middle third of the oligomer.

6. A set of oligomers according to claim 3, comprising at least one oligomer for at least one of the CpG dinucleotides of one of the sequences according to Seq. ID No.1 to Seq. ID No.4.

7. A set of oligomers according to claim 6, comprising at least one oligomer for each of the CpG dinucleotides of one of Seq. ID No.1 to Seq. ID No.4.

8. A set of at least two oligonucleotides as recited in claim 2 which can be used as primer oligonucleotides for the amplification of DNA sequences of one of Seq. ID No.1 to Seq. ID No.4 or segments thereof.

9. A set of oligonucleotides as recited in claim 8, characterised in that at least one oligonucleotide is bound to a solid phase.

10. A set of oligomer probes for detecting the cytosine methylation status and/or single nucleotide polymorphisms (SNPs) in the chemically pretreated DNA of the gene for CD24 according to one of Seq. ID No.1 to Seq. ID No.4, comprising at least ten of the oligomers according to one of claims 2 to 5.

11. A method for manufacturing an arrangement of different oligonucleotides and/or PNA-oligomers (array) fixed to a carrier material for analysing diseases associated with the methylation status of the gene CD24, wherein at least one oligomer according to any of claims 2 to 5 is coupled to a solid phase.

12. An arrangement of different oligonucleotides and/or PNA-oligomers (array), according to any of claims 2 to 5.

13. An array of different oligonucleotide- and/or PNA-oligomer sequences as recited in claim 12, characterised in that these are arranged on a plane solid phase in the form of a rectangular or hexagonal lattice.

14. The array as recited in any of claims 12 or 13, characterised in that the solid phase surface is composed of silicon, glass, polystyrene, aluminium, steel, iron, copper, nickel, silver, or gold.

15. A DNA-chip for analysing diseases associated with the methylation status of the gene CD24, comprising at least one nucleic acid according to one of the preceding claims.

16. A kit and/or diagnostic agent, comprising a bisulfite (=disulfite, hydrogen sulfite) reagent as well as at least one oligomer according to any of claims 2 to 5, optionally together with suitable auxiliary compounds and additives.

17. A method for ascertaining genetic and/or epigenetic parameters of the gene CD24 by analysing cytosine methylations, comprising the following steps:
a) in a genomic DNA sample, cytosine bases which are unmethylated at the 5-position are converted, by chemical treatment, to uracil or another base which is dissimilar to cytosine in terms of hybridisation behaviour;
b) fragments of the chemically pretreated genomic DNA are amplified using sets of primer oligonucleotides according to claim 8 or 9 and a polymerase, the amplificates carrying a detectable label;
c) the amplificates are hybridised to a set of oligonucleotides and/or PNA probes according to the claims 2 to 5, or to an array according to one of the claims 12 to 14; and
d) the hybridised amplificates are subsequently detected.

18. The method as recited in claim 17, wherein the chemical treatment is carried out by means of a solution of a bisulfite, hydrogen sulfite or disulfite.

19. The method as recited in one of the claims 17 or 18, characterised in that more than ten different fragments having a length of 100-2000 base pairs are amplified.

20. The method as recited in one of the claims 17 to 19, characterised in that the amplification of several DNA segments is carried out in one reaction vessel.

21. The method as recited in one of the claims 17 to 20, characterised in that the polymerase is a heat-resistant DNA polymerase.

22. The method as recited in claim 21, characterised in that the amplification is carried out by means of the polymerase chain reaction (PCR).

23. The method as recited in one of the claims 17 to 22, characterised in that the labels of the amplificates are fluorescence labels, radionuclides or detachable molecule fragments having a typical mass which are detected in a mass spectrometer.

24. The method as recited in one of the claims 17 to 23, characterised in that the amplificates or fragments of the amplificates are detected in the mass spectrometer.

25. The method as recited in claim 24, characterised in that the produced fragments have a single positive or negative net charge for better detectability in the mass spectrometer.

26. The method as recited in claim 24 or 25, characterised in that detection is carried out and visualised by means of matrix assisted laser desorption/ionisation mass spectrometry (MALDI) or using electron spray mass spectrometry (ESI).

27. The method as recited in one of the claims 17 to 26, characterised in that the genomic DNA is obtained from cells or cellular components which contain DNA, sources of DNA comprising, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and combinations thereof.

28. Use of a nucleic acid according to claim 1 for the diagnosis of diseases associated with CD24.

29. The use of the oligomers according to one of the claims 2 to 5 or an array thereof according to one of the claims 12 to 14 or a kit according to claim 16 for the diagnosis of a disease associated with CD24 by analysing methylation patterns of the gene CD24.

30. The use according to claim 29 for the diagnosis of cancerous diseases, for example leukaemia, lung cancer or the nasopharyngal karcinoma, multiple myeloma, reactive arthritis, spleen-lymphoma, Waldenstrom macroglobulinemia, Epstein-Barr-Virus induced syndrome and/or infantile spinal muscle atrophy.

31. Method for the diagnosis and/or prognosis of disadvantageous events for patients or individuals, wherein the data which are obtained with a method according to any of the preceding claims are assigned to a disease associated with CD24.

32. Use of the data which are obtained with a method according to any of the preceding claims for the diagnosis and/or prognosis of disadvantageous events for patients or individuals, wherein said disadvantageous events are related to methylation patterns of the gene CD24.
